(19) United States
(12) Patent Application Publication
(10) Pub. No.: US 2010/0082064 A1
(43) Pub. Date: Apr. 1, 2010

(54) METHOD FOR COATING METALLIC SURFACES OF MEDICAL DEVICES WITH AN ANTI-INFECTIVE AGENT

(76) Inventors: Iksoo Chun, Princeton, NJ (US); Thoppil Mathew John, Easton, PA (US); Raymond S. Shissias, Iselin, NJ (US); Bruce A. Lowenhaupt, Hillsborough, NJ (US); Uri Herzberg, Bridgewater, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/241,503

(22) Filed: Sep. 30, 2008

(51) Int. Cl. A61B 17/70; A61L 27/54

(52) U.S. Cl. 606/246; 427/2.1; 427/2.26

(57) ABSTRACT

A method of coating metallic surfaces of medical devices with an anti-infective agent is disclosed. Specifically, a method of providing a discontinuous coating of triclosan on the metallic surface of a medical device is disclosed.

### FIELD OF THE INVENTION

This invention relates to a method of coating metallic surfaces of a medical device with an anti-infective coating, and a medical device having a coated metallic surface.

### BACKGROUND OF THE INVENTION

Implanted medical devices in general, and orthopedic devices in particular, are often contaminated with infectious bacteria introduced either during trauma or during the surgical procedure. Once the infectious bacteria are introduced onto or around the medical device, a biofilm can form on or in the medical device. Such biofilms, which are known in this art, are composed of bacteria and an extracellular matrix that is secreted by the bacteria. It is generally recognized that such a biofilm is extremely resistant to antibiotics and various treatment protocols. As such, the generally accepted treatment is to remove the implanted device and either clean or sterilize it and re-implant, or replace it with another sterile device.

Coating the device with an antimicrobial agent to provide an anti-infective surface is a possible solution to assist in addressing this problem. Typically, a conventional antimicrobial agent is coated onto a surface of the medical device in combination with a conventional carrier, such as absorbable polymers, nonabsorabable polymers, and other carriers. Although the carriers enable the anti-infective agent to be coated onto a surface, such carriers may present several issues, such as substantially increasing the thickness of the device, providing a smooth coating that causes instability of the device, and delamination of the coating from the device. In lieu of a carrier, functional groups may be added to the surface of the medical device. The antimicrobial agent may be tethered to the surface of the device using such functional groups. Functional groups on the device surface may present other issues associated with the bio-compatibility of such groups, including the possibility that such groups may possess active biological effects once the antimicrobial agent leaves the device. Therefore, there is a need in this art for alternative methods of applying anti-infective coatings onto the metallic surfaces of medical devices.

### SUMMARY OF THE INVENTION

Accordingly, metallic medical devices having an anti-infective coating of triclosan, and methods of making the same are described and disclosed. One aspect of the present invention is a coated medical device. The medical device consists of a medical device having a metallic surface. The surface may be present on a part or section of the device, or may be present on substantially all of the device. The device has a discontinuous coating of triclosan or another anti-infective agent on the metallic surface.

Yet another aspect of the present invention is a method for coating a metallic surface of a medical device with an coating of an anti-infective coating, in particular triclosan. In this method a coating solution is provided. The coating solution consists of triclosan or another anti-infective agent and a solvent, preferably an organic solvent. A medical device is provided having a metallic surface. The metallic surface of the medical device is electrostatically sprayed with the coating solution, thereby providing a discontinuous coating of triclosan on the metallic surface of the medical device.

These and other aspects and advantages of the present invention will become more apparent from the following description and accompanying drawings.

### BRIEF DESCRIPTION OF THE DRAWINGS

FIGS. 1a-d are SEM micrographs of titanium spinal rods: a control rod (uncoated) and rods coated with triclosan. FIG. 1a shows an example of a control rod (uncoated). FIG. 1b shows a rod coated with triclosan using ethanol as a solvent. FIG. 1c shows a rod coated with triclosan using acetone as the solvent. FIG. 1d shows a rod coated with triclosan using methylene chloride as a solvent.

FIGS. 2a-d are SEM micrographs of coated and uncoated stainless steel rods following wash with warm Lactated Ringer's Solution (LRS) to explore the stability of the coating during surgical wash conditions. FIG. 2a shows an uncoated stainless steel rod prior to wash in LRS. FIG. 2b shows a triclosan coated stainless steel rod, prior to wash in LRS. FIG. 2c shows a triclosan coated stainless steel rod after wash in LRS. FIG. 2d shows an uncoated stainless steel rod, after wash in LRS.

FIG. 3 is a graph illustrating triclosan elution from a coated spinal rod, when placed in phosphate buffered saline.

FIG. 4 is a perspective view of a spinal rod having a discontinuous triclosan coating of the present invention, a pedicle screw and a set screw.

### DETAILED DESCRIPTION OF THE INVENTION

Described herein is a medical device having a metallic surface coated with an anti-infective agent and methods of applying the coating. Preferably, the medical device is made from a biocompatible metal as further described herein, and has metallic surfaces. It is also possible that the medical device can be a combination or composite of metal and non-metallic components and has one or more metallic surfaces. In addition, the medical device may be a non-metal such as ceramic or polymer, with a metal coating deposited on one or more surfaces of the device. In any case, the present invention provides for a method of coating an anti-infective coating on the metallic surfaces of a medical device. The anti-infective agent is triclosan. The metallic medical device is comprised of a conductive metal suitable for medical use including, but not limited to stainless steel, titanium, bioabsorbable metals, metal alloys, and the like. In one embodiment, the conductive metal is stainless steel or titanium. As previously mentioned, the metallic medical device may be made completely of a conductive metal or it may be a medical device made of a non-metallic core material and coated with a metal such that the device has a metallic surface, or the device may be a composite of non-metallic and metallic materials. Exemplary non-metallic materials include, but are not limited to absorbable polymers, nonabsorbable polymers, ceramics, polymer/polymer composites, polymer/ceramic composites, polymer absorbable metal composites, and the like. The term metallic surfaces as used herein is defined to mean both exterior metal surfaces, as well as interior or internal metal surfaces.

The term anti-infective agent is defined to mean a material capable of acting against infection, by inhibiting the spread of an infectious agent or by killing the infectious agent outright. Anti-infective is a general term that includes conventional anti-bacterials, antibiotics, anti-fungals, anti-protozoans, anti-parasitics and antivirals, and accordingly, infectious agents include known infectious agents such as bacteria, fungi, viruses, and parasites, etc.

Triclosan is a known, conventional anti-infective agent that is useful in preventing infections by acting as a bactericide, and may have other anti-infective properties. Triclosan is sold under the trade name IRGASAN® (CIBA Specialty Chemicals Corporation, Tarrytown, N.Y.). Triclosan is coated on the metallic surfaces of medical device using the process of the present invention in an amount sufficiently effective to prevent infection from occurring in the area around and about the medical device before and after implantation by acting as an anti-infective agent. Triclosan may be coated onto the metallic surfaces of a medical device, for example, in an amount of from about 0.01 mg/cm² to about 10 mg/cm². In one embodiment, triclosan is present on the medical device in an amount of from about 0.01 mg/cm² to about 0.1 mg/cm². In another embodiment, triclosan is present on the medical device in an amount of about 0.03 mg/cm².

The metallic surfaces of the medical device are coated by electrostatically spraying a solution of triclosan in a suitable, conventional organic solvent. The solution is prepared by dissolving a sufficiently effective amount of triclosan in the solvent at room temperature. Suitable organic solvents include, but are not limited to acetone, methylene chloride, and alcohols, such as ethanol, propanol and isopropanol. In one embodiment, the solvent includes, but is not limited to acetone, ethanol, and methylene chloride. In another embodiment, the solvent is acetone. For example, a solution of triclosan useful in the practice of the present invention is prepared by dissolving the triclosan in the amount of about 0.5 g of triclosan/100 mL of solvent to about 10 g of triclosan/100 mL of solvent. In one embodiment, the solution of triclosan is prepared by dissolving the triclosan in the amount of about 0.5 g of triclosan/100 mL of solvent to about 5 g of triclosan/100 mL of solvent. In another embodiment, the solution of triclosan is prepared by dissolving the triclosan in the amount of about 1 g of triclosan/100 mL of solvent. Although not preferred, and if one skilled in the art were willing to accept any attendant disadvantages, the solution may be prepared with water.

Utilizing the method of the present invention, the metallic surface or surfaces of the medical device are coated using conventional electrostatic spraying techniques. The medical device is secured to a chuck, the metallic surface or surfaces are grounded, and the device is rotated about the chuck at a sufficiently effective rotational velocity. The rotational speed is typically in the range of from about 1 rpm to about 500 rpm. In one embodiment, the rotational speed is in the range of from about 10 rpm to about 100 rpm. A sufficiently effective electrical charge is then applied to the triclosan solution, typically in the range of from about 5 kV to about 20 kV. The solution is then injected at a sufficiently effective rate and a charged mist of the solution is attracted to the grounded device to produce a coating of the triclosan solution. The solution is injected at a sufficiently effective flow rate in the range of typically from about 0.5 mL/hour to about 18 mL/hour. The solution injection time is sufficiently effective and is typically about 1 min to about 4 min. The injector is held at a distance of from about 2 cm to about 20 cm from the device while spraying. The electrostatic spraying may be done at a suitable temperature, for example at room temperature or in a heated environment of up to about 60° C. The residual solvent was removed from the coated rods by air-drying in a ventilated hood. Alternatively, the solvent may be removed by conventional means such as vacuum drying, drying in an inert atmosphere, and the like.

This process of the present invention provides a discontinuous coating of triclosan on the metallic surface or surfaces of the medical device. Triclosan is present on the device in the form of crystals. This coating does not substantially change the dimensions of the device. The coating also provides areas of bare metal for ease of securing the device and ensuring stability, such as preventing axial rotation or slipping in the case of spinal rods. The amount of the coating applied will be sufficiently effective to protect the tissue in the vicinity of the device from becoming infected, to prevent localized infections from spreading or becoming systemic, or to provide an adequate amount of a biologically active ingredient to achieve a desired biological outcome. Such agents can be anti-infective agents, agents that enhance tissue integration and agents that improve surgical outcomes. The coating amount, for example, will range from about 0.01 mg/CM² coated areas to about 10 mg/CM² coated areas.

It is advantageous to have a discontinuous coating of triclosan on the metal surfaces medical device rather than a continuous coating. This is so since metal to metal contact of the rod with the pedicle screw is important in the case of spinal rods to maintain the mechanical integrity of the device. In the case of bone plates or other medical devices that are going to be coated, it will be important to maintain device-bone integration or device-tissue attachment and as such maintaining areas that are “bare” is important.

The method of the present invention for coating a metallic surface of a medical device with an anti-infective agent such as triclosan described herein is useful for any metallic medical device that can become infected during surgery, since the presence of triclosan will preclude the bacteria from colonizing the device and forming a biofilm on the metallic surfaces of the device. An example of particularly preferred metallic medical devices which may have their metal surface or surfaces coated using the process of the present invention are those devices used in the vicinity of a hematoma, in surgeries where soft tissue has been extensively manipulated such as, for example, in fixing bones following trauma and in spinal fixation devices where metal-to-metal contact is important to maintain mechanical stability. The process of the present invention is useful for coating metallic surfaces of any metallic or composite medical devices, however it is particularly useful for orthopedic devices. Exemplary metallic medical devices that may have their metal surfaces coated with an anti-infective coating such as triclosan include, but are not limited to spinal rods, sternal wires, bone pins, bone plates, bone screws, bone replacement devices, such as knees, hips, and joints, and spinal cages. In one embodiment, the metallic medical devices are sternal wires. In another embodiment the metallic medical device is a spinal rod.

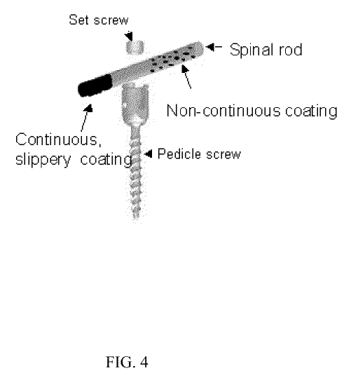

A metallic spinal rod device that may be coated using the process of the present invention is illustrated in FIG. 4. The spinal rod 10 is seen to have an elongated cylindrical body 20 having opposed ends 30 and 40, and adjacent end section 35 and 45. The body 20 is seen to have exterior surface 50. The section 52 of surface 50 in end section 35 is seen to have discontinuous anti-infective coating 60. The section 54 of surface 50 in end section 45 is seen to have continuous slippery coating 70. Slippery coating 70 is a conventional biocompatible coating and may consist of a biocompatible polymer and an anti-infective agent. Pedicle screw 100 is seen to have elongated body 110 having distal pointed end 114 and proximal end 117. Bone engaging conventional screw threads 120 are seen to extend from the surface 119 of elongated body 110. Extending from the proximal end 114 is the mounting head 130 The interior 132 of mounting head 130 has interior screw threads 134 for mating with screw threads 152 on set screw 150, when set screw 150 is received within interior 132. Head 130 is also seen to have U-shaped opening 136 for receiving a section of spinal rod 10. Spinal rod 10 is mounted to pedicle screw 100 in the following manner. The spinal rod 10 is placed in the U-shaped opening of mounting head 130, then the set screw 150 is attached to the mounting head 130 via the interior screw threads 134 engaging the screw threads 152 on the set screw and tightened sufficiently to retain the spinal rod 10 in mounting head 130.

The coating method and device having a coated surface of the present invention have many advantages, including: slip resistance and mechanical stability comparable to bare metal or rod alone, increased slip resistance and mechanical stability over continuous polymer coatings encapsulating an anti-infective agent, and the drug coating alone inhibits bacterial growth and infection in the area surrounding the implant without needing a polymer for controlled release of the drug.

The following examples are illustrative of the principles and practice of this invention, although not limited thereto. Numerous additional embodiments within the scope and spirit of the invention will become apparent to those skilled in the art once having the benefit of this disclosure.

### EXAMPLES

### Example 1

### Method of Coating Spinal Rods

Titanium spinal rods that were 2 in long and 5.5 mm in diameter were obtained from DePuy Spine, Inc. (Raynham, Mass.). Three 1% (w/v) solutions of triclosan were prepared in ethanol, acetone, and methylene chloride, respectively, by adding 1 g of triclosan in 100 mL of solvent at room temperature with stirring. The spinal rods were coated with the triclosan solutions using a conventional electrostatic spray coater. The spray coater was manufactured by Terronics Development Corporation, Elwood, Ind. Dart Nozzle with Small Set Back. The spinal rod was secured in the chuck and grounded. The coating conditions were as follows: distance of rod from injection nozzle=7 cm; voltage applied to the coating solution=12 Kv; rotational speed=34 rpm; and injection rate of 4 mL/hour. The each coating solution was applied for either 2 min or 4 min. After applying the coating to the rods, the solvent was removed by air drying under ambient conditions in a laboratory fume hood. The weight of the bare rod was subtracted from the weight of the coated rod to determine the total weight of coating applied to the rod under these conditions. The coating weights for 3 rods were averaged and are shown in Table 1 below.

TABLE 1

| | Solvent | | |
|---|---|---|---|
| | | | Methylene |
| Time coated | Ethanol | Acetone | Chloride |
| 2 minutes | 0.743 ± 0.023 | 0.907 ± .087 | 1.043 ± .055 |
| 4 minutes | 1.503 ± .0.625 | 1.887 ± .051 | 2.243 ± .098 |

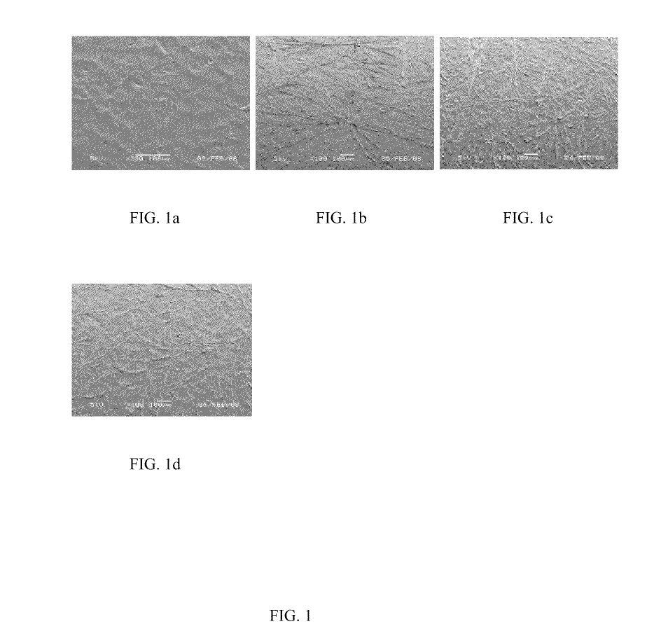

Scanning electron micrographs (SEMs) were taken of the coated and uncoated rods using a (JSM-5900LV) SEM (JEOL USA, Inc., Peabody, Mass.). The micrographs shown in FIGS. 1a-d. show the uncoated rod (FIG. 1a) in comparison to rods coated with triclosan using ethanol as a solvent (FIG. 1b), using acetone as the solvent (FIG. 1c), and using methylene chloride as a solvent (FIG. 1d). While the methylene chloride was the most efficient solvent for coating, all subsequent work was performed with acetone as the solvent, since methylene chloride is not an environmentally friendly solvent.

### Example 2

### Coating Stability after Lactated Ringer's Solution Wash

During a surgical procedure, a surgeon will typically wash the surgical area with a solution to clear the area from loose tissue, blood clots and reduce the chance of infection. A common wash solution is Lactated Ringers Solution (LRS). It is therefore important that the coated rods coated with the method of the present invention do not lose the coating when subjected to such wash conditions. One coated titanium rod and one coated stainless steel rod, each coated by the method described in Example 1 using acetone as the solvent and a 2 min spray time, as well as uncoated rods (titanium and stainless steel) were tested for stability of the triclosan coating. The rods were subjected to wash and incubation using LRS. The procedure used was as follows: after recording the weight of the rods, each rod was individually placed in a 15 ml-capacity polypropylene centrifuge tube. The rod was then rinsed with LRS (pre-warmed to 37° C.) by pouring the LRS solution into the tube and decanting after 15 seconds. Fresh LRS (pre-warmed to 37° C.) was subsequently added to the tube and the tube was immediately placed in a shaking water-bath (37° C.; 55 rev/min) and allowed to shake for 2 minutes. The rods were taken out and air-dried in a laboratory fume hood for 30 min at room temperature and the weights were recorded. The net weight difference was calculated. The results are summarized in Tables 2a and 2b.

TABLE 2a
Triclosan coated rods washed in Lactated Ringer's Solution

| | Pre-wash | Post-wash | Difference |
|---|---|---|---|
| Sample ID | wt (mg) | wt (mg) | (mg) |
| 3664-56-SS-6 | 10733.72 | 10733.82 | 0.10 |
| 3664-56-T-6 | 5744.81 | 5744.88 | 0.07 |
| Mean | | | 0.09 |

TABLE 2b
Uncoated rods washed in Lactated Ringer's Solution

| | | Post- | |
|---|---|---|---|
| | Pre-wash | wash wt | Difference |
| Sample ID | wt (mg) | (mg) | (mg) |
| uncoated SS control | 10462.61 | 10462.78 | 0.17 |
| uncoatedTitanium control | 5621.32 | 5621.50 | 0.14 |
| Mean | | | 0.16 |

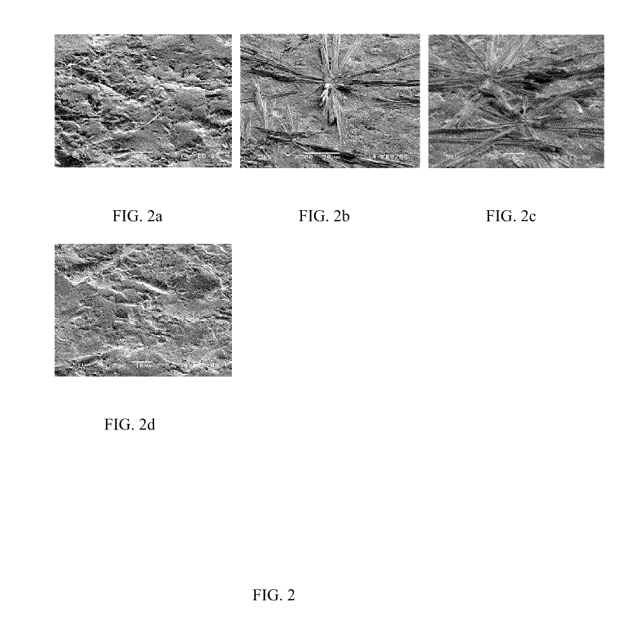

As can be seen from the tables, there was no loss of material from the rods in either group. The additional weight can be explained by the fact that some of the solutes present in the LRS may have been added to the rods' surface. The weight increase is small and can also be explained as being within the experimental error of the balance. Scanning electron micrographs (SEMs) were taken of the coated and uncoated rods using a (JSM-5900LV) SEM (JEOL USA, Inc., Peabody, Mass.). The micrographs shown in FIGS. 2a-d. indicate that the morphology of the coated rod did not change following the wash with LRS solution.

### Example 3

### Mechanical Stability Testing

In almost all orthopedic devices, mechanical stability of the fixed construct is important. This is especially important in spinal rods. The objective of implanted spinal rods is to transfer mechanical load from the spine to the rod. The rods accept spinal loads through pedicle screws, and the rod-screw mechanical interfaces are critical to the function of the spinal rod. In many pedicle screw and rod assemblies, the screws mate with the rod at the screw's head. Typically, the screw head has a saddle feature to accept the rod. After the rod is placed, a set screw is tightened onto the rod thereby capturing the rod within the screw head. The rod is compressed between the pedicle screw head and the set screw. In this configuration, the rod cannot rotate, translate, or twist with respect to the pedicle screw Screw rod assemblies are subjected to axial slip testing (defined by ASTM F1717) to test for the potential effects of the coating on the mechanical stability of the rod-pedicle screw construct. Axial slip testing was conducted by attaching a pedicle screw to one rod end, securing the other rod end in a vice, then placing a load on the screw head where the load vector is parallel to the long-axis of the rod.

The pedicle screw was attached to the rod using manufacturer's instructions and specifications. The set-screw was applied using a torque driver set to 9 N*m or 80 inch-pounds (as per manufacturer's instructions). After the pedicle screw was attached near one rod end (at least 4 mm of rod must protrude from the screw head to eliminate so-called “end effects”), the other end of the rod was fixed in a rigid vice/clamp/chuck to create a mechanical cantilever condition (the rod cannot rotate, translate, or slide within the cantilever vice). The cantilevered rod was then placed on a rigid mechanical stage (anvil) and aligned with the mechanical crosshead (hammer). The crosshead applied a load to the pedicle screw head. The direction of the load was parallel to the long-axis of the cantilevered spinal rod. The crosshead was under displacement control of 25 mm/minute. Crosshead displacement was terminated after 0.5 mm of crosshead travel, a sudden change in mechanical load, or after it was clear that the pedicle screw head was slipping on the rod. The peak load was determined by analyzing the load-displacement curve or by measuring the yield-load at 2% displacement offset using the axial slip stiffness determined by the linear region of the load-displacement curve.

The test was repeated for 5 rods each that were uncoated and dry, coated and dry, and coated and wet. All rods were coated as described in example 1 using acetone as the solvent and a 2 min coating time. The wet rods were soaked in warm (38° C.) saline (0.9% NaCl) overnight. Table 3 shows the average displacement force for each condition tested.

TABLE 3

| | Uncoated - | Triclosan | Triclosan Coated - |
|---|---|---|---|
| Rod Treatment | Dry | Coated - Dry | wet |
| Slip Resistance | 1358.8 ± 27.61 | 1731.6 ± 46.81 | 1210 ± 81.17 |
| (Newton) | | | |

The coated rods provided the same stability as uncoated rods. There was no statistical difference in the slip resistance between the uncoated and coated rods, even when wet.

The further slip testing was performed as described above to compare the uncoated rods with rods coated with a polymer used to deliver the triclosan. Titanium rods were coated with poly(epsilon-caprolactone-co-lactide) (PCL/PLA) containing triclosan. The polymer coating solution was prepared by dissolving 5 g of PCL/PLA polymer (PURAC Biochem BV Gorinchen, Netherlands) and 2 g of triclosan in 100 mL methylene chloride.

Titanium spinal rods that were 2 in long and 5.5 mm in diameter were obtained from DePuy Spine (Raynham, Mass.). Coating was performed using an electrostatic spray coater. The spinal rod was secured in the chuck and the coating conditions were as follows: distance of rod from injection nozzle=7.5 cm; voltage applied to the coating solution=12.5 Kv; rotational speed=32 rpm; and injection rate of 6 mL/hour. The coating solution was applied for 30 seconds. Slip testing and wetting of the rods was performed as described above. Table 4 shows the average displacement force for each condition (N=5) tested.

TABLE 4

| | | | Polymer | Polymer |
|---|---|---|---|---|
| | Uncoated - | Uncoated - | Coated - | Coated - |
| Treatment | Dry | Wet | Dry | Wet |
| Slip | 1347.2 ± 22.72 | 1462.8 ± 61.42 | 982.2 ± 115.1 | 838.1 ± |
| Resistance | | | | 230.8 |

The slip resistance decreased by a statistically significant amount for the polymer coated rods in comparison to the uncoated rods either wet or dry. However, there was no significant difference in the triclosan coated rods in comparison to uncoated rods.

### Example 4

### Zone of Inhibition Test (ZOI)

A common test to evaluate the efficacy of anti microbial agents is the zone of inhibition test. The test uses a 10 cm Petri dish that is loaded with agar containing bacteria. A sample is placed in the middle of the dish and as the anti microbial agent elutes from the device the area of killed bacteria is assessed. The rods coated with triclosan were tested for efficacy against Staph. aureus bacteria. Rods were coated with triclosan as described in Example 1 using acetone as the solvent and with a 2 min coating time. The rods were placed in Petri dishes that were inoculated with Staphylococcus aureus bacteria with at least 1.6×10⁵ CFU/mL of agar solution and incubated for 24 hours at 37° C. The rods were then transferred to a fresh 10 mm Petri dish that was also inoculated with bacteria.

The coated rods were effective in not only reducing the bacteria in a recognized assay of bacterial growth (Zone of inhibition: ZOI), the bacteria were completely eliminated from the test dish. This was confirmed since the coated rods completely eliminated the bacteria inoculated in an agar Petri dish compared with non-coated rods. When the coated rods were transferred to a newly inoculated dish at 24 hours time points. The bacteria were again completely eliminated at 48 and 72 hours in comparison to uncoated rods.

### Example 5

### Controlled Release of Triclosan from the Rods

This example was conducted to provide data concerning the kinetics of triclosan elution from the coated rods. The release of triclosan from the coated rods was performed in a phosphate buffered saline solution. Rods were coated as described in Example 1 using acetone as the solvent and with a 2 minute coating time. Phosphate buffered saline (PBS) was prepared by dissolving Sigma PBS powder (P-3813) in an appropriate volume of purified water to obtain a pH=7.4. Four 50-mL capacity glass tubes were set up and 25 mL of PBS was added to each of the tubes. One coated rod sample was placed into each of these tubes. The tubes were then placed in a shaking water-bath set at 30 rev/min and 37° C. The full content of the PBS (25 ml) was removed from each tube at 1 hr, 1 day, 4 day, 7 day, 16 day, 21 day and 24 day time points. Each time the PBS was removed was it was replaced with 25 ml of fresh buffer. All collected samples were stored in glass vials at 4° C. for HPLC analysis of triclosan content.

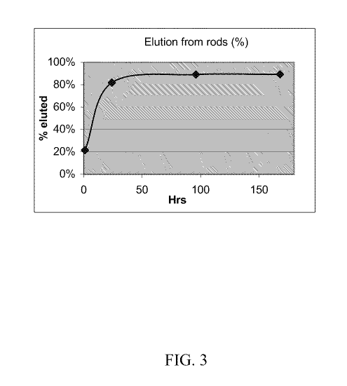

As illustrated in FIG. 3, the triclosan indeed eluted from the coated rod. A burst release of about 20% of the triclosan was observed followed by controlled release over the next four days. Furthermore, a complete elution was achieved by 96 hours. The additional 10% not accounted for was most likely trapped on the walls of the glass tubes due to the multiple solution transfers. No additional elution was observed between the 4 days time point (96 hours) and the following time points.

### Example 6

### Method of Coating a Bone Plate

In addition to spinal rods other metallic medical devices may be coated using the methods of the present invention described herein. For example, it may be useful to coat bone plates with triclosan. Using the method described herein, a medical device such as a bone plate can be coated with triclosan. Bone plates must be in close proximity to the bone and are prone to infection. Such plates, if coated with a polymer carrier, are likely to lose the proximity to the bone when the polymer erodes. Indeed, it is possible to coat the bone plate on the side that does not meet the bone, yet it is preferable that the all sides of the plate are coated with an anti-microbial agent to maintain infection free bone fixation. Using the described method in Example 1, a bone plate is fixed in a chuck, attached to a ground electrode, and the nozzle from which a triclosan solution is attached to a charged electrode. The triclosan solution will be in the range of 0.1-10% and preferably in the range of 1-2%. The solution is then injected at a specific rate, of 1000-0.1 ml/hour and preferably at a rate of 10-1 ml/hour. The bone plate is either rotated or it moves back and forth in front of the mist created by the injected triclosan. Following adequate time to allow enough coating on the bone plate while avoiding too much triclosan being deposited on the bone plate the bone plate is removed from the coating procedure. It is important to remove the bone plates soon enough such that the plate maintains areas of bare metal—not coated with triclosan.

### Example 7

### Surgical Procedure for Spinal Rod Placement

The coated rods produced by the methods described above in Example 1 can be used in surgery to stabilize the spinal column of a patient. A conventional spinal stabilization procedure provides back stability to patients suffering from degeneration of the vertebrae, or any other part of the spinal column such as the intervertebral discs, trauma, scoliosis and other back instability. In such surgeries, the patient is anesthetized by conventional techniques. The area is prepared, an incision followed by tissue retraction and further tissue separation are made to access the spine. The spine is manipulated by the surgeon to the desired position. Stabilization is provided by placing pedicle screws in the bony part of the spinal column, while attaching the spinal rods to the pedicle screws. A tight connection between the pedicle screws and the spinal rods is achieved by tightening the set-screw (see FIG. 4), prevents the spinal column from “springing back” to its pre-surgical position. The incision is then closed upon completion of the procedure.

Although this invention has been shown and described with respect to detailed embodiments thereof, it will be understood by those skilled in the art that various changes in form and detail thereof may be made without departing from the spirit and scope of the claimed invention.

We claim:

1. A coated medical device, comprising,
a medical device having a metallic surface; and,
an anti-infective coating on said metallic surface, wherein the coating is discontinuous.

2. The medical device of claim 1, where the coating is present in an amount of about 0.01 mg/cm² to about 10 mg/cm².

3. The medical device of claim 1, where the coating is present in an amount of about 0.01 mg/cm² to about 0.1 mg/cm².

4. The medical device of claim 1, where the coating has is present in an amount of about 0.03 mg/cm² to about 0.1 mg/cm².

5. The device of claim 1, wherein the surface is an exterior surface.

6. The device of claim 1, wherein the surface is an interior surface.

7. The device of claim 1, wherein the device has a plurality of metallic surfaces.

8. The device of claim 1, wherein the device comprises a metal spinal rod.

9. The device of claim 1, wherein the anti-infective agent comprises triclosan.

10. A method for coating a metallic surface of a medical device with triclosan, comprising the steps of:
providing a coating solution comprising an anti-infective agent and a solvent;
providing a medical device having a metallic surface; and,
electrostatically coating the metallic surface of the medical device with the coating solution, thereby, providing a discontinuous coating of the anti-infective agent on the metallic surface of the medical device.

11. The method of claim 10, where the anti-infective coating is present in an amount of about 0.01 mg/cm² to about 10 mg/cm².

12. The method of claim 10, where the anti-infective coating is present in an amount of about 0.01 mg/cm² to about 0.1 mg/cm².

13. The method of claim 10, where the anti-infective coating is present in an amount of about 0.03 mg/cm² to about 0.1 mg/cm².

14. The method of claim 10, wherein the surface is an exterior surface.

15. The method of claim 10, wherein the surface is an interior surface.

16. The method device of claim 10, wherein the device has a plurality of metallic surfaces.

17. The method of claim 10, wherein the device comprises a metal spinal rod.

18. The method of claim 10, wherein the anti-infective agent comprises triclosan.

19. The method of claim 10, wherein the solvent comprises an organic solvent.

20. The method of claim 10, wherein the device is rotated during the electrostatic coating step.

21. The device of claim 1, wherein the anti-infective agent is selected from the group consisting of antibacterials, antivirals, anti-fungals, and combinations thereof.

22. The method of claim 10, wherein the anti-infective agent is selected from the group consisting of antibacterials, antivirals, anti-fungals, and combinations thereof.
